(19) United States
(12) Patent Application Publication
(10) Pub. No.: US 2022/0023630 A1
(43) Pub. Date: Jan. 27, 2022

(54) Apparatus for Strengthening Muscle Contraction (e.g., Cardiac Muscle Contraction) Using Electric Fields

(71) Applicants: Yoram PALTI, Haifa (IL); Rafael BEYAR, Haifa (IL)

(72) Inventors: Yoram PALTI, Haifa (IL); Rafael BEYAR, Haifa (IL)

(21) Appl. No.: 17/378,998

(22) Filed: Jul. 19, 2021

Related U.S. Application Data
(60) Provisional application No. 63/054,383, filed on Jul. 21, 2020.

(51) Int. Cl. A61N 1/362; A61N 1/365

(52) U.S. Cl. CPC A61N 1/3627; A61N 1/3625; A61N 1/365

(57) ABSTRACT

An apparatus for improving the cardiac function and cardiac output of a patient comprises a waveform generator that generates alternating voltage pulses, a controller to control the timing of the pulses, and electrodes that deliver the alternating voltage pulses to the patient's body. The alternating voltage pulses induce a field of alternating current pulses within the patient's body. As the pulses pass through a cardiac ventricle (or atrium), they increase the concentration of Ca²⁺ at the appropriate cardiomyocyte sites, and thereby increase the strength and duration of the ventricular (or atrial) contractions. In alternative embodiments, the electric field may be used to strengthen the contractions of non-cardiac muscle (e.g., skeletal muscle).

### CROSS REFERENCE TO RELATED APPLICATIONS

This application claims the benefit of U.S. Provisional Application 63/054,383, filed Jul. 21, 2020, which is incorporated herein by reference in its entirety.

### BACKGROUND

About 6.5 million adults in the United States have suffered from some form of heart failure. A heart failure diagnosis does not necessarily mean the heart has stopped beating. To the contrary—heart failure occurs any time the heart cannot pump enough blood and oxygen to support other organs in the body. This can occur when the contractions of a person's heart muscles are not strong enough and/or have insufficient duration and synchronization to pump the volume of blood that is needed to support whatever activity the person is doing.

### SUMMARY OF THE INVENTION

One aspect of the invention is directed to a first apparatus for improving cardiac function in a patient. The first apparatus comprises a controller, a waveform generator, and a plurality of electrodes. While operating under the control of the controller, the waveform generator generates an output of alternating voltage pulses. The plurality of electrodes is electrically coupled to the output of the waveform generator, and is configured to deliver the alternating voltage pulses to a region of the patient's chest. The controller is programmed to control the waveform generator so that the alternating voltage pulses generated by the waveform generator are timed to coincide with a portion of a cardiac cycle.

In some embodiments of the first apparatus, the controller receives a timing parameter from an external source. Responsive to receiving the timing parameter from the external source, the controller (i) determines a timeframe for the waveform generator to generate the alternating voltage pulses based at least in part on the timing parameter, and (ii) sends a signal to the waveform generator that causes the waveform generator to generate the alternating voltage pulses during the timeframe. Optionally, in these embodiments, the external source comprises an ECG.

Optionally, in the ECG embodiments described in the previous paragraph, the controller is programmed to control the waveform generator so that the alternating voltage pulses generated by the waveform generator are timed to coincide with a portion of a cardiac cycle when the patient's left ventricle contracts or the patient's cardiac atriums contract. Optionally, in the ECG embodiments described in the previous paragraph, the controller is programmed to control the waveform generator so that the alternating voltage pulses generated by the waveform generator are timed to begin slightly before a portion of a cardiac cycle when the patient's left ventricle contracts or the patient's cardiac atriums contract.

In some embodiments of the first apparatus, the controller receives a timing parameter from an external source. Responsive to receiving the timing parameter from the external source, the controller (i) determines a timeframe for the waveform generator to generate the alternating voltage pulses based at least in part on the timing parameter, and (ii) sends a signal to the waveform generator that causes the waveform generator to generate the alternating voltage pulses during the timeframe, and the external source comprises a pacemaker. Optionally, in these embodiments, the timing parameter comprises a beginning time for a pacer pulse generated by the pacemaker.

In some embodiments of the first apparatus, the alternating voltage pulses delivered to the region of the patient's chest induces in the region a field of alternating current pulses.

In some embodiments of the first apparatus, the alternating voltage pulses delivered to the region of the patient's chest induces in the region a field of alternating current pulses, and the plurality of electrodes are arranged on or below the skin of the patient's chest so that a portion of the alternating current pulses in the field will pass through a left ventricle within the patient's chest.

In some embodiments of the first apparatus, the alternating voltage pulses delivered to the region of the patient's chest induces in the region a field of alternating current pulses, and the plurality of electrodes are arranged on or below the skin of the patient's chest so that most of the alternating current pulses in the field will pass through a left ventricle within the patient's chest.

In some embodiments of the first apparatus, the alternating voltage pulses delivered to the region of the patient's chest induces in the region a field of alternating current pulses, and the alternating current pulses in the field has a frequency greater than 10 kHz.

In some embodiments of the first apparatus, the alternating voltage pulses delivered to the region of the patient's chest induces in the region a field of alternating current pulses. The controller sends a signal to the waveform generator that causes the waveform generator to generate two or more trains of alternating voltage pulses for one cardiac cycle of the patient, thereby inducing in the region of the patient's chest a field comprising two or more trains of alternating current pulses for said one cardiac cycle of said patient. Optionally, in these embodiments, each train in said two or more trains of alternating current pulses in the field has a duration in a range of 2-200 ms. Optionally, in these embodiments, each train in said two or more trains of alternating voltage pulses has an amplitude in a range of 0.1-20 volts.

In some embodiments of the first apparatus, the controller receives a needs parameter from an external source. Responsive to receiving the needs parameter for the patient from the external source, the controller (i) determines an adjusted timeframe for the waveform generator to generate the alternating voltage pulses based at least in part on the needs parameter, and (ii) sends a signal to the waveform generator that causes the waveform generator to generate the alternating voltage pulses during the adjusted timeframe.

In some embodiments of the first apparatus, the controller receives a needs parameter from an external source, and the controller issues a command to the waveform generator that causes the waveform generator to generate the alternating voltage pulses based on the needs parameter.

Some embodiments of the first apparatus further comprise a sensor. In these embodiments, the controller receives a sensor input parameter collected by the sensor, and the controller issues a command to the waveform generator that causes the waveform generator to generate the alternating voltage pulses based on the sensor input parameter.

In some embodiments of the first apparatus, the apparatus further comprises a manual input device, the controller receives a manual input parameter collected at the manual input device, and the controller issues a command to the waveform generator that causes the waveform generator to generate the alternating voltage pulses based on the manual input parameter.

Another aspect of the invention is directed to a second apparatus for increasing a contraction force of at least one muscle in a subject. The second apparatus comprises a controller, a waveform generator, and a plurality of electrodes. While operating under the control of the controller, the waveform generator generates an output of alternating voltage pulses. The plurality of electrodes are electrically coupled to the output of the waveform generator, and are configured to deliver the alternating voltage pulses to a vicinity of the at least one muscle. The controller is programmed to control the waveform generator so that the alternating voltage pulses generated by the waveform generator are timed to coincide with a time when increasing the contraction force of the at least one muscle is desired.

In some embodiments of the second apparatus, the controller receives a timing parameter from an external source. In these embodiments, responsive to receiving the timing parameter from the external source, the controller (i) determines a timeframe for the waveform generator to generate the alternating voltage pulses based at least in part on the timing parameter, and (ii) sends a signal to the waveform generator that causes the waveform generator to generate the alternating voltage pulses during the timeframe.

Another aspect of the invention is directed to a method for increasing a contraction force of at least one muscle in a subject. The method comprises positioning a plurality of electrodes on or in the subject's body at respective positions selected such that when an alternating voltage is applied between the plurality of electrodes, an alternating electric field will be induced within the at least one muscle. The method also comprises applying alternating voltage pulses between the plurality of electrodes at a plurality of times when increasing the contraction force of the at least one muscle is desired, wherein the alternating voltage pulses cause alternating current pulses to pass through the at least one muscle and increase an amount of free intracellular Ca2+ ions available to the at least one muscle. And the method also comprises, at the end of each of the plurality of times, discontinuing the alternating voltage pulses, so as to cause a reduction in the amount of free intracellular Ca2+ ions available to the at least one muscle.

### BRIEF DESCRIPTION OF THE DRAWINGS

Various embodiments are described in detail below with reference to the accompanying drawings. In these drawings:

FIG. 1 shows a high-level schematic diagram of an apparatus for treating cardiac patients configured to operate in accordance with an embodiment of the present invention.

FIG. 2 shows a waveform timing diagram illustrating, by way of example, a series of alternating current pulses produced by the apparatus shown in FIG. 1.

FIG. 3 shows a top view of a transverse section of a female thorax to illustrate, by way of example, an arrangement of the waveform generator and two electrodes placed on the chest wall of a cardiac patient.

### DESCRIPTION OF THE PREFERRED EMBODIMENTS

Normally, intracellular Ca²⁺ concentration ([Ca²⁺]ᵢₙ) is very low in all living cells, including cardiomyocytes. During the resting (diastole) state, [Ca²⁺]ᵢₙ is 10⁻⁷ molar. Following ventricle muscle excitation and generation of action potentials, L-type Ca²⁺ channels open, resulting in an influx of Ca²⁺ ions into the cardiomyocytes along the electro-chemical gradient. At this point, a unique process occurs. First, a massive number of Ca²⁺ ions are released from the sarcoplasmic reticulum and the released Ca²⁺ ions enter each cell cytoplasm. As a result, the concentration of Ca²⁺ in the medium surrounding the contracting mechanism of the cells (actin and myosin filaments) increases significantly (to over 10⁻⁶ molar), thus taking a critical part in muscle contraction. The strength of the muscle contraction is a function of the Ca²⁺ concentration ([Ca²⁺]ᵢₙ) in the cells. The influx of Ca²⁺+ ions (counter-balanced by outward K+ currents) sustains depolarization of the cell membrane, thereby creating a “plateau phase” of the action potential and a corresponding long-lasting contraction typical to cardiac muscle. As the plateau phase progresses, the intracellular Ca²⁺ (associated with the contraction) is pumped back to the sarcoplasmic reticulum by the ATP-dependent Ca²⁺ pump (SERCA), and extruded outside the cell by the Na—Ca exchanger (NCX). This causes the Ca²⁺ concentration in the cells to drop to 10⁻⁷ molar, which effectively halts the contraction so that the muscle relaxes.

More forceful and longer-lasting contractions of cardiac muscle can be induced by increasing the amount of free intracellular Ca²⁺ ions that are available during a specific timeframe within the cardiac cycle. And this increase in the amount of free intracellular Ca²⁺ ions can be brought about by applying an alternating current to the cardiac muscle. Likewise, inducing the cardiac muscle to stop contracting and relax long enough for the heart to collect an adequate amount of blood in preparation for the next contraction can be promoted by reducing the amount of free intracellular Ca²⁺ ions available outside of that specific timeframe.

In general, embodiments of the present invention provide an apparatus for treating cardiac patients, comprising a waveform generator that generates alternating voltage pulses, a controller to control the timing of the waveform generated by the waveform generator, and a set of electrodes, electrically coupled to the output of the waveform generator, which are configured to deliver the alternating voltage pulses to the patient's body.

The alternating voltage pulses delivered to the patient's body by the set of electrodes induces within the patient's body a field of alternating current pulses, referred to as “Cardiac Treating Field pulses” (or “CTF pulses”). The set of electrodes are placed in locations on or below the skin of the patient's chest so that a significant portion of the current in the CTF pulses will pass through the mass of the patient's ventricles and atria. As the CTF pulses pass through the heart, mainly through the left ventricle, the concentration of Ca²⁺ surges at the cardiomyocyte sites, and thereby increases the amplitude (strength) and duration of cardiac contractions.

The electrodes are driven by the waveform generator, and the waveform generator is controlled by the controller. The controller controls the timing for the beginning and the end of each pulse of alternating voltage generated by the waveform generator (e.g., based on a variety of different cardiac-related triggers, external parameters, sensor measurements and manual inputs) by issuing appropriate commands to the waveform generator. The controller may also determine the amplitude, frequency and duration of the alternating voltage pulses based on these same triggers, external parameters, sensor measurements and manual inputs.

At an appropriate first time during each cardiac cycle, the controller causes the waveform generator to start generating the alternating voltage pulses, which causes alternating current pulses (i.e., CTF pulses) to pass through the mass of the cardiac muscles, thereby increasing the amount of free intracellular Ca²⁺ ions available to the cardiomyocytes, which will increase the strength of the ventricles' contraction. Then, at an appropriate second time during each cardiac cycle or as determined on the basis of the output of an appropriate sensor, the controller causes the waveform generator to stop generating the alternating voltage pulses, which eliminates the induced CTF pulses passing through the ventricles, thereby reducing the amount of free intracellular Ca²⁺ ions available to the cardiomyocytes. This reduction in the number of free intracellular Ca²⁺ ions available to the cardiomyocytes causes the ventricles to relax and stay relaxed long enough to fill up with an adequate amount of blood for the next contraction. The increased strength and duration of the cardiac contractions can improve the output of the patient's heart, and thereby increases the amount of blood and oxygen available to support other organs in the patient's body.

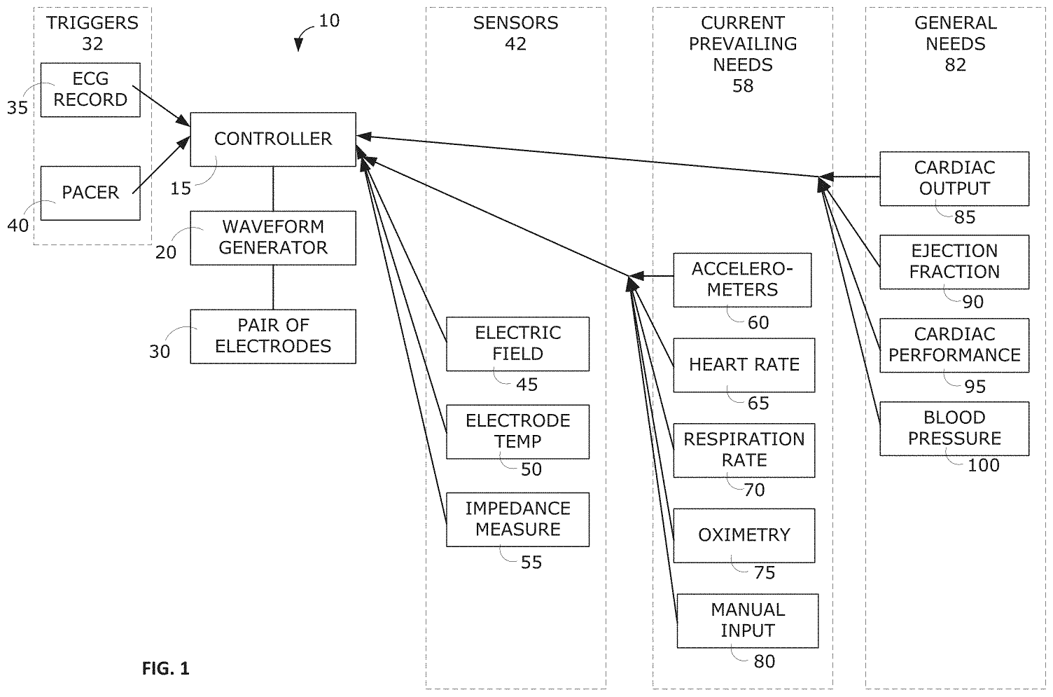

Turning now to the figures, FIG. 1 shows a high-level schematic diagram of an apparatus 10 for treating cardiac patients in accordance with an embodiment of the present invention. As shown in FIG. 1, the apparatus 10 comprises a controller 15, a waveform generator 20, and at least two electrodes 30. In some embodiments, the controller 15 is implemented using a commercially available microprocessor or a microcontroller, operatively coupled to RAM and nonvolatile memory. The nonvolatile memory stores program instructions that are executed by the microprocessor or microcontroller, and execution of those instructions causes the microprocessor or microcontroller to perform the steps described herein. A variety of alternative approaches for implementing the controller 15 will be apparent to persons skilled in the relevant arts, including but not limited to using a hardwired controller or a microcoded controller.

In some embodiments, the waveform generator 20 includes a low-level waveform generator that generates an intermediate signal, and an amplifier configured to amplify that intermediate signal. In alternative embodiments, a single-stage waveform generator that is capable of generating an output with sufficiently high voltage to induce the appropriate amount of current to flow through the heart is used. The CTF pulses are generated by the waveform generator 20 operating under control of the controller 15. The CTF pulses are initiated on the basis of one or more triggers 32 that the controller 15 receives from an external source or external device, such as an ECG 35 for the patient, or a pacer 40 in cases where the patient uses a pacemaker, etc.

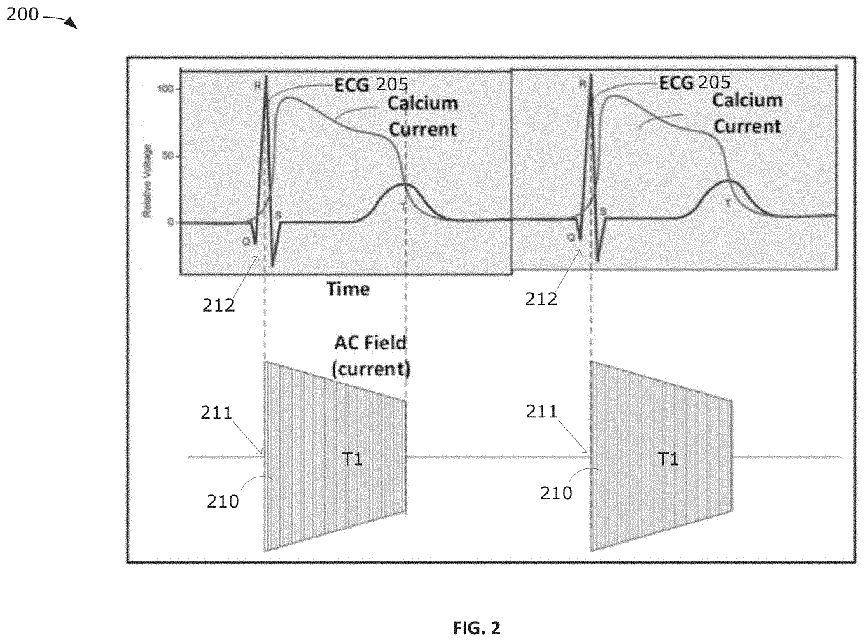

FIG. 2 shows a waveform timing diagram 200 illustrating, by way of example, a series of CTF pulses 210, each comprising a set of alternating current fields produced by the apparatus shown in FIG. 1. A typical ECG 205 is also shown in FIG. 2. As shown in FIG. 2, the CTF pulses 210 are timed to coincide with the portion of the cardiac cycle when the left ventricle contracts. This may be accomplished by inducing the CTF pulses 210 to start whenever a QRS complex of the ECG 205 is detected, and ending the CTF pulses 210 at the apex of the T wave. In alternative embodiments, it may be accomplished by inducing the CTF pulses 210 to start whenever a pacemaker's pacing pulse is detected.

If previous pulse timings are available, the timing of the upcoming QRS complex or pacing pulse can be predicted, in which case the CTF pulses 210 may be timed to precede the Q deflection of the QRS complex or the pacing pulse by a short time (e.g., 5-50 ms). By starting the CTF pulses 210 slightly before (e.g., 5-50 ms) contraction of the ventricle is expected to begin, the amount of Ca²⁺ ions will already be raised at the instant the contraction begins, which will increase the force of the contraction. The application of the CTF pulses 210 may continue throughout the entire contraction process, when the Ca²⁺ ions are beneficial. But the CTF pulses 210 should not be extended to the relaxation period, so that the Ca²⁺ ions will not interfere with the relaxation of the ventricle.

When pulses of alternating electric voltage are applied to the human body with electrodes, the voltage pulses induce a field of corresponding pulses of alternating electric current in the areas of the body adjacent to the electrodes. The amplitudes of the alternating electric current pulses 210 are determined by the body's impedance. The field distribution, and thus the current distribution, is determined by the geometry and the relative impedances of the different system components. The frequency of the alternation of the CTF pulses 210 is high enough to avoid stimulating the cardiac muscle or any of the other muscles and nerves that fall within the electric field generated. Preferably, the frequency is greater than 10 kHz.

Each CTF pulse 210 comprises at least one group or “wave train” T1 of high frequency pulses or waves. However, it may consist of additional wave trains (T2 . . . Tn) within the framework of the cardiac cycle. Suitably, the characteristics of the trains (T2 . . . Tn) of the CTF pulses 210 are determined by the controller 15 based on a combination of the patient's current prevailing needs 58 and/or general needs 82, both of which may serve as inputs to the operation of the controller 15. The train durations range preferably is 2-200 ms and the amplitudes are preferably in the range of 1-200 Volts.

The potential need for different waveform characteristics at different stages of the cardiac cycle stems from the different roles of cellular Ca²⁺ on contraction power, etc. at these stages. There are at least three such stages: (1) the increase in [Ca²⁺]ᵢₙ during the membrane depolarization associated with excitation, (2) the large increase in [Ca²⁺]ᵢₙ that maintains the long duration muscle contraction, and (3) the decrease in [Ca²⁺]ᵢₙ during repolarization, which leads to muscle relaxation, the integrity of which is an essential part of cardiac diastole.

The CTF pulses 210 are timed and adjusted in duration and amplitude according to the measured and predicted patient's needs. These needs may include, for example, general needs 82, examples of which are depicted in FIG. 1, as well as the current prevailing needs 58, examples of which are also shown in FIG. 1. Among other things, these inputs of current prevailing needs 58 may include, for example, measurements provided by accelerometers 60, current heart rate 65, respiration rate 70, oximetry 75 and manual input 80. The general needs 82 may include a variety of standard or typical cardiac-related values, such as cardiac output 85, ejection fraction 90, cardiac performance 95 (which may include the desired contraction power) and blood pressure 100, as shown in FIG. 1. Expected future needs (not shown in the figures) may also be fed into the controller 15 by manual input 80. Examples of expected future needs include, for example, physical exertion, such as stair or hill climbing, exposure to extreme weather conditions, expected excitement, sports, etc. The controller 15 uses the expected future needs, the current prevailing needs 58 and the general needs 82 of the patient to determine the various CTF pulse 210 characteristics.

In addition to the above, the CTF pulse 210 characteristics may also be controlled based on inputs from the various deployed sensors 42 that monitor a variety of different relevant parameters that the CTF pulse 210 generation may change or depend upon. These relevant parameters may include, for example, electric field sensors 45, electrode temperature sensors 50 and an impedance measuring sensor 55.

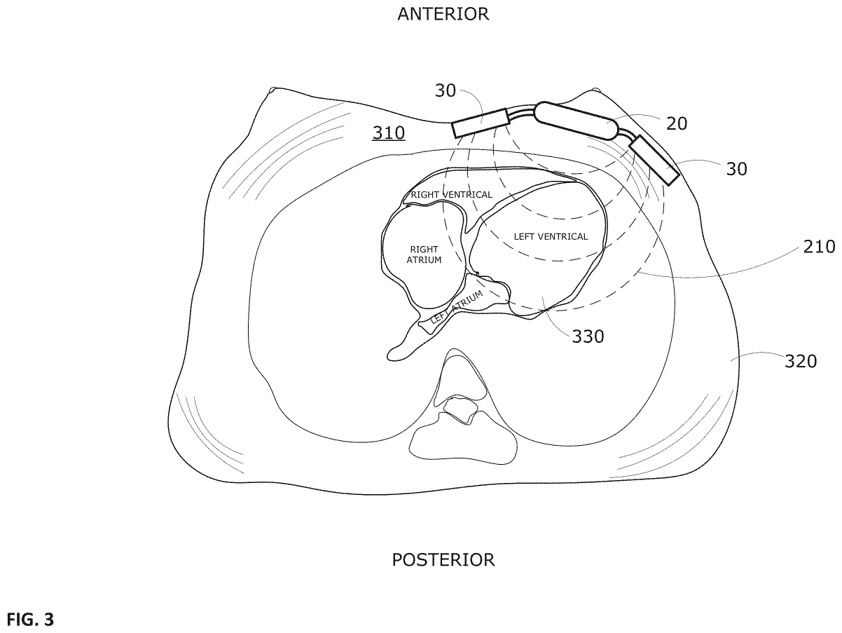

FIG. 3 illustrates, by way of example, an arrangement of the waveform generator 20 and the electrodes 30 components as they may be placed on the chest wall 310 of a female cardiac patient in one embodiment of the invention. As shown in FIG. 3, two electrodes 30 are placed on the chest wall 310 of the patient to provide the alternating voltage pulses so that most of the CTF pulses 210 of alternating electric current induced inside the patient's body 320 by the alternating voltage pulses will pass through the mass of the left ventricle 330. To improve electrical contact between the electrodes 30 and the skin, a suitable hydrogel may be applied directly to the skin of the chest wall 310 before attaching the electrodes 30.

Devices configured to deliver CTF pulses 210 in accordance with embodiments of the present invention may have a number of different configurations. For example, in some embodiments, the device may comprise a battery-operated patch removably attached to the chest wall of the patient with a suitably non-toxic adhesive, as depicted in FIG. 3. In another embodiment (not shown in the figures), the device may comprise a plurality of chest electrodes driven by a waveform generator configured to be carried in a breast pocket or hip pocket of the patient's clothing. In still another embodiment, the device may comprise a component of a pacemaker configured to generate and deliver the appropriate alternating voltage pulses to the patient's chest (in addition to the conventional cardiac stimulating pulses generated by the pacemaker). Although FIG. 3 shows two electrodes 30 attached to the skin of the patient, it will be appreciated that, in certain embodiments, the device may comprise epicardial electrodes implanted during cardiac surgery. The set of electrodes may also include three, four or more electrodes, depending on the situation.

In the embodiments described above, electric fields that are timed to coincide with ventricular contractions are applied to the ventricles in order to strengthen the contraction of cardiomyocytes located in the ventricles. But in alternative embodiments, a similar approach may be used to strengthen the contraction of cardiomyocytes located in the right and/or left atriums. In these alternative embodiments, the electric fields are applied to the atrial walls by repositioning the electrodes, and the timing of the electric fields is modified with respect to the embodiments described above. More specifically, the strength of atrial contraction is augmented by applying the AC electric fields to the atrial walls during a window of time that runs between the beginning of the P wave of an ECG and the R wave of the ECG's QRS complex. This may be accomplished by inducing the CTF pulses 210 to start whenever a P wave of the ECG 205 is detected. Applying the electric fields during this window of time may also advantageously help control atrial fibrillation and other cardiac arrhythmias.

If previous pulse timings are available, the timing of the upcoming P wave can be predicted, in which case the CTF pulses 210 may be timed to precede the P wave by a short time (e.g., 5-50 ms). By starting the CTF pulses 210 slightly before (e.g., 5-50 ms) contraction of the ventricle is expected to begin, the amount of Ca²⁺ ions will already be raised at the instant the contraction begins, which will increase the force of the contraction. The application of the CTF pulses 210 may continue throughout the entire contraction process, when the Ca²⁺ ions are beneficial. But the CTF pulses 210 should not be extended to the relaxation period, so that the Ca²⁺ ions will not interfere with the relaxation of the atria.

In the embodiments described above, electric fields are used to strengthen the contractions of cardiac muscles. But in alternative embodiments, electric fields may be used to strengthen the contractions of other types of muscles including skeletal muscles and smooth muscles (e.g., muscles in the GI tract, bladder, uterus and vascular system). The role of calcium in initiating contraction is similar (although not identical) in these types of muscles. Improving muscular contraction can improve the mechanical performance of both normal subjects and subjects suffering from muscular and neuromuscular diseases such as: Neuromuscular disorders, such as muscular dystrophies, multiple sclerosis (MS), amyotrophic lateral sclerosis (ALS); Autoimmune diseases, such as Graves' disease, myasthenia gravis, and Guillain-Barré syndrome; Thyroid conditions, such as hypothyroidism and hyperthyroidism; Electrolyte imbalances, such as hypokalemia, hypomagnesemia, and hypercalcemia; stroke, herniated disc, chronic fatigue syndrome (CFS), hypotonia, peripheral neuropathy, neuralgia, polymyositis, or chronic muscle inflammation.

Note that in the context of cardiac muscles described above, synchronization/triggering of the field induced [Ca²⁺] change to an ECG is preferably done on a repetitive basis for each heartbeat in a series of successive heartbeats. But the timing will be different in the context of other muscles, depending on the particular muscle whose contraction is being strengthened. In some contexts, it will be appropriate to time the contraction-strengthening field to coincide with nerve or muscle electric activity. In some embodiments, the contraction-strengthening fields may be used to increase the strength of skeletal muscle contractions to, for example, help a person walk or to lift a heavier load than he or she might otherwise be able to lift. The application of the electric field in these embodiments may be initiated automatically using sensors to detect the natural contraction of the relevant muscles, and then rapidly applying the electric field to boost the strength of the contraction of the relevant muscles. In other embodiments, the contraction-strengthening fields may be used to help a person empty their bladder or control sphincters, etc. A wide variety of alternatives can be readily envisioned, depending on the nature of the particular muscle whose contraction strength is being boosted. The demand detection or determination may be similar to that currently used in cardiac pacemakers, for example intensity of movement detection by accelerators, oxygen/CO₂ levels.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but it has the full scope defined by the language of the following claims, and equivalents thereof.

What is claimed is:

1. An apparatus for improving cardiac function in a patient, the apparatus comprising:
a controller;
a waveform generator that, while operating under the control of the controller, generates an output of alternating voltage pulses; and
a plurality of electrodes, electrically coupled to the output of the waveform generator, configured to deliver the alternating voltage pulses to a region of the patient's chest,
wherein the controller is programmed to control the waveform generator so that the alternating voltage pulses generated by the waveform generator are timed to coincide with a portion of a cardiac cycle.

2. The apparatus of claim 1, wherein:
the controller receives a timing parameter from an external source, and
responsive to receiving the timing parameter from the external source, the controller (i) determines a timeframe for the waveform generator to generate the alternating voltage pulses based at least in part on the timing parameter, and (ii) sends a signal to the waveform generator that causes the waveform generator to generate the alternating voltage pulses during the timeframe.

3. The apparatus of claim 2, wherein the external source comprises an ECG.

4. The apparatus of claim 3, wherein the controller is programmed to control the waveform generator so that the alternating voltage pulses generated by the waveform generator are timed to coincide with a portion of a cardiac cycle when the patient's left ventricle contracts.

5. The apparatus of claim 3, wherein the controller is programmed to control the waveform generator so that the alternating voltage pulses generated by the waveform generator are timed to coincide with a portion of a cardiac cycle when the patient's cardiac atriums contract.

6. The apparatus of claim 3, wherein the controller is programmed to control the waveform generator so that the alternating voltage pulses generated by the waveform generator are timed to begin slightly before a portion of a cardiac cycle when the patient's left ventricle contracts.

7. The apparatus of claim 3, wherein the controller is programmed to control the waveform generator so that the alternating voltage pulses generated by the waveform generator are timed to begin slightly before a portion of a cardiac cycle when the patient's cardiac atriums contract.

8. The apparatus of claim 2, wherein the external source comprises a pacemaker.

9. The apparatus of claim 8, wherein the timing parameter comprises a beginning time for a pacer pulse generated by the pacemaker.

10. The apparatus of claim 1, wherein the alternating voltage pulses delivered to the region of the patient's chest induces in the region a field of alternating current pulses.

11. The apparatus of claim 10, wherein the plurality of electrodes are arranged on or below the skin of the patient's chest so that a portion of the alternating current pulses in the field will pass through a left ventricle within the patient's chest.

12. The apparatus of claim 10, wherein the plurality of electrodes are arranged on or below the skin of the patient's chest so that most of the alternating current pulses in the field will pass through a left ventricle within the patient's chest.

13. The apparatus of claim 10, wherein the alternating current pulses in the field has a frequency greater than 10 kHz.

14. The apparatus of claim 10, wherein the controller sends a signal to the waveform generator that causes the waveform generator to generate two or more trains of alternating voltage pulses for one cardiac cycle of the patient, thereby inducing in the region of the patient's chest a field comprising two or more trains of alternating current pulses for said one cardiac cycle of said patient.

15. The apparatus of claim 14, wherein each train in said two or more trains of alternating current pulses in the field has a duration in a range of 2-200 ms.

16. The apparatus of claim 14, wherein each train in said two or more trains of alternating voltage pulses has an amplitude in a range of 0.1-20 volts.

17. The apparatus of claim 1, wherein:
the controller receives a needs parameter from an external source, and
responsive to receiving the needs parameter for the patient from the external source, the controller (i) determines an adjusted timeframe for the waveform generator to generate the alternating voltage pulses based at least in part on the needs parameter, and (ii) sends a signal to the waveform generator that causes the waveform generator to generate the alternating voltage pulses during the adjusted timeframe.

18. The apparatus of claim 1, wherein:
the controller receives a needs parameter from an external source, and
the controller issues a command to the waveform generator that causes the waveform generator to generate the alternating voltage pulses based on the needs parameter.

19. The apparatus of claim 1, wherein:
the apparatus further comprises a sensor,
the controller receives a sensor input parameter collected by the sensor, and
the controller issues a command to the waveform generator that causes the waveform generator to generate the alternating voltage pulses based on the sensor input parameter.

20. The apparatus of claim 1, wherein:
the apparatus further comprises a manual input device,
the controller receives a manual input parameter collected at the manual input device, and
the controller issues a command to the waveform generator that causes the waveform generator to generate the alternating voltage pulses based on the manual input parameter.

21. An apparatus for increasing a contraction force of at least one muscle in a subject, the apparatus comprising:
a controller;
a waveform generator that, while operating under the control of the controller, generates an output of alternating voltage pulses; and
a plurality of electrodes, electrically coupled to the output of the waveform generator, configured to deliver the alternating voltage pulses to a vicinity of the at least one muscle,
wherein the controller is programmed to control the waveform generator so that the alternating voltage pulses generated by the waveform generator are timed to coincide with a time when increasing the contraction force of the at least one muscle is desired.

22. The apparatus of claim 21, wherein:
the controller receives a timing parameter from an external source, and
responsive to receiving the timing parameter from the external source, the controller (i) determines a timeframe for the waveform generator to generate the alternating voltage pulses based at least in part on the timing parameter, and (ii) sends a signal to the waveform generator that causes the waveform generator to generate the alternating voltage pulses during the timeframe.

23. A method for increasing a contraction force of at least one muscle in a subject, the method comprising:
positioning a plurality of electrodes on or in the subject's body at respective positions selected such that when an alternating voltage is applied between the plurality of electrodes, an alternating electric field will be induced within the at least one muscle;
applying alternating voltage pulses between the plurality of electrodes at a plurality of times when increasing the contraction force of the at least one muscle is desired, wherein the alternating voltage pulses cause alternating current pulses to pass through the at least one muscle and increase an amount of free intracellular Ca²⁺ ions available to the at least one muscle; and
at the end of each of the plurality of times, discontinuing the alternating voltage pulses, so as to cause a reduction in the amount of free intracellular Ca²⁺ ions available to the at least one muscle.
